(19) United States
(12) Patent Application Publication
(10) Pub. No.: US 2021/0347728 A1
(43) Pub. Date: Nov. 11, 2021

(54) 4,5-DIHYDROXY-2-(4-METHYLBENZYL)ISOPHTHALONITRILE SOLVATES AND CRYSTALLINE FORMS THEREOF

(71) Applicant: Orion Corporation, Espoo (FI)

(72) Inventor: David DIN BELLE, Espoo (FI)

(21) Appl. No.: 17/262,249

(22) PCT Filed: Jul. 24, 2019
(86) PCT No.: PCT/EP2019/069962

§ 371 Date: Jan. 22, 2021

Related U.S. Application Data
(60) Provisional application No. 62/703,077, filed on Jul. 25, 2018.

(51) Int. Cl. C07C 255/53; C07C 253/30

(52) U.S. Cl. CPC C07C 255/53; C07B 2200/13; C07C 253/30

(57) ABSTRACT

The present disclosure relates to 4,5-dihydroxy-2-(4-methylbenzyl) isophthalonitrile solvates, including hydrates, and crystalline forms thereof and to methods for preparing said solvates and crystalline forms thereof.

### FIELD OF THE INVENTION

The present disclosure relates to 4,5-dihydroxy-2-(4-methylbenzyl)isophthalonitrile solvates, including hydrates, and crystalline forms thereof and to methods for preparing said solvates and crystalline forms thereof.

### BACKGROUND OF THE INVENTION

The compound 4,5-dihydroxy-2-(4-methylbenzyl)isophthalonitrile of formula (I) has been disclosed in WO 2013/175053.

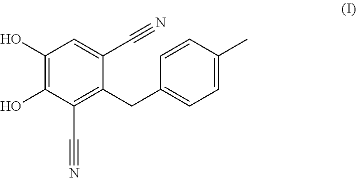 [SMILES: Cc1ccc(Cc2c(C#N)cc(O)c(O)c2C#N)cc1]

4,5-Dihydroxy-2-(4-methylbenzyl)isophthalonitrile is a catechol O-methyltransferase (COMT) inhibitor. COMT inhibitors have been shown to be effective in clinical use for the treatment of Parkinson's disease as an adjunct to levodopa therapy. COMT inhibitors have also been indicated to be useful in the treatment of, for example, hypertension, heart failure and depression (U.S. Pat. No. 5,446,194) as well as inhibitors for the prevention of diabetic vascular dysfunctions (WO 98/27973). COMT inhibitors have also been disclosed as being useful for treating or controlling pain (WO 01/68083) as well as for treating restless legs syndrome (RLS), which is also known as Ekbom's syndrome (WO 2006/051154).

Several properties, such as solubility, bioavailability after oral administration, melting point, chemical stability, hygroscopicity, physical stability and processability, of a drug substance are often dependent on the crystalline form and the solvent possibly incorporated in the crystal lattice. The crystalline form of the substance and the solvent possibly incorporated in the crystal lattice may affect also the dissolution of the drug product.

Polymorphic and solvatomorphic control is thus vital in the manufacture of pharmaceutical products. The importance is further emphasized in the case of substances possessing extensive polymorphism and/or solvatomorphism.

### SUMMARY OF THE INVENTION

In one embodiment, the present disclosure relates to a compound which is crystalline form II of 4,5-dihydroxy-2-(4-methylbenzyl)isophthalonitrile monohydrate.

In one embodiment, the present disclosure relates to a compound which is crystalline form I of 4,5-dihydroxy-2-(4-methylbenzyl)isophthalonitrile anhydrate.

In one embodiment, the present disclosure relates to a compound which is crystalline form IV of 4,5-dihydroxy-2-(4-methylbenzyl)isophthalonitrile anhydrate.

In one embodiment, the present disclosure relates to a compound which is crystalline form III of 4,5-dihydroxy-2-(4-methylbenzyl)isophthalonitrile anhydrate.

In one embodiment, the present disclosure relates to a compound which is crystalline form V of 4,5-dihydroxy-2-(4-methylbenzyl)isophthalonitrile presumably methanol solvate.

In one embodiment, the present disclosure relates to a compound which is crystalline form VI of 4,5-dihydroxy-2-(4-methylbenzyl)isophthalonitrile monohydrate.

In one embodiment, the present disclosure relates to a compound which is crystalline form VII of 4,5-dihydroxy-2-(4-methylbenzyl)isophthalonitrile presumably methanol solvate.

In one embodiment, the present disclosure relates to a compound which is crystalline form VIII of 4,5-dihydroxy-2-(4-methylbenzyl)isophthalonitrile family of isomorphous solvates T8 presumably ethanol/propan-2-ol and T8a presumably propan-2-ol.

In one embodiment, the present disclosure relates to a compound which is crystalline form IX of 4,5-dihydroxy-2-(4-methylbenzyl)isophthalonitrile presumably ethanol solvate.

In one embodiment, the present disclosure relates to a compound which is crystalline form X of 4,5-dihydroxy-2-(4-methylbenzyl)isophthalonitrile presumably ethanol solvate.

In one embodiment, the present disclosure relates to a compound which is crystalline form XI of 4,5-dihydroxy-2-(4-methylbenzyl)isophthalonitrile presumably ethanol solvate.

Furthermore, methods for preparing said compounds and pharmaceutical dosage forms comprising said compounds are provided.

Form II monohydrate is stable within a reasonable humidity range, enables adequate polymorphic and solvatomorphic control in the manufacture of pharmaceutical products and is suitable for use in wet granulation.

### BRIEF DESCRIPTION OF THE DRAWINGS

FIG. 1: X-ray powder diffractogram of crystalline form II of 4,5-dihydroxy-2-(4-methylbenzyl)isophthalonitrile monohydrate obtained in Example 1.

FIG. 2: X-ray powder diffractogram of crystalline form I of 4,5-dihydroxy-2-(4-methylbenzyl)isophthalonitrile anhydrate obtained in Example 2.

FIG. 3: X-ray powder diffractogram of crystalline form IV of 4,5-dihydroxy-2-(4-methylbenzyl)isophthalonitrile anhydrate obtained in Example 3.

FIG. 4: X-ray powder diffractogram of crystalline form III of 4,5-dihydroxy-2-(4-methylbenzyl)isophthalonitrile anhydrate obtained in Example 4.

FIG. 5: X-ray powder diffractogram of crystalline form V of 4,5-dihydroxy-2-(4-methylbenzyl)isophthalonitrile presumably methanol solvate obtained in Example 5.

FIG. 6: X-ray powder diffractogram of crystalline form VI of 4,5-dihydroxy-2-(4-methylbenzyl)isophthalonitrile monohydrate obtained in Example 6.

FIG. 7: X-ray powder diffractogram of crystalline form VII of 4,5-dihydroxy-2-(4-methylbenzyl)isophthalonitrile presumably methanol solvate obtained in Example 7.

FIG. 8: X-ray powder diffractograms of crystalline form VIII of 4,5-dihydroxy-2-(4-methylbenzyl)isophthalonitrile family of isomorphous solvates T8 presumably ethanol/propan-2-ol and T8a presumably propan-2-ol obtained in Example 8.

FIG. 9: X-ray powder diffractogram of crystalline form IX of 4,5-dihydroxy-2-(4-methylbenzyl)isophthalonitrile presumably ethanol solvate obtained in Example 9.

FIG. 10: X-ray powder diffractogram of crystalline form X of 4,5-dihydroxy-2-(4-methylbenzyl)isophthalonitrile presumably ethanol solvate obtained in Example 10.

FIG. 11: X-ray powder diffractogram of crystalline form XI of 4,5-dihydroxy-2-(4-methylbenzyl)isophthalonitrile presumably ethanol solvate obtained in Example 11.

### DETAILED DESCRIPTION OF THE INVENTION

It has now been found that 4,5-dihydroxy-2-(4-methylbenzyl)isophthalonitrile possesses extensive polymorphism and solvatomorphism.

In one embodiment, the present disclosure relates to a compound which is crystalline form II of 4,5-dihydroxy-2-(4-methylbenzyl)isophthalonitrile monohydrate, wherein the compound has at least peaks at 2θ of about 6.1° and 11.2° in an X-ray powder diffractogram.

In one embodiment, the present disclosure relates to a compound which is crystalline form II of 4,5-dihydroxy-2-(4-methylbenzyl)isophthalonitrile monohydrate, wherein the compound has at least peaks at 2θ of about 6.1° and 14.7° in an X-ray powder diffractogram.

In one embodiment, the present disclosure relates to a compound which is crystalline form II of 4,5-dihydroxy-2-(4-methylbenzyl)isophthalonitrile monohydrate, wherein the compound has at least peaks at 2θ of about 6.1°, 11.2°, 14.7°, 16.2°, 21.8°, 24.0°, 26.2° and 26.9° in an X-ray powder diffractogram.

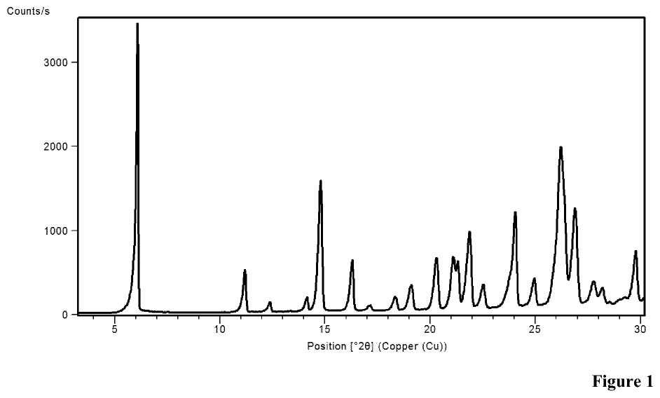

In one embodiment, the present disclosure relates to a compound which is crystalline form II of 4,5-dihydroxy-2-(4-methylbenzyl)isophthalonitrile monohydrate, wherein the compound is characterized by an X-ray powder diffractogram substantially as illustrated in FIG. 1.

In one embodiment, the present disclosure relates to a compound which is crystalline form II of 4,5-dihydroxy-2-(4-methylbenzyl)isophthalonitrile monohydrate, wherein the compound contains less than 25% by weight of other 4,5-dihydroxy-2-(4-methylbenzyl)isophthalonitrile solvates and crystalline forms thereof.

In one embodiment, the present disclosure relates to a compound which is crystalline form II of 4,5-dihydroxy-2-(4-methylbenzyl)isophthalonitrile monohydrate, wherein the compound contains less than 10% by weight of other 4,5-dihydroxy-2-(4-methylbenzyl)isophthalonitrile solvates and crystalline forms thereof.

In one embodiment, the present disclosure relates to a compound which is crystalline form II of 4,5-dihydroxy-2-(4-methylbenzyl)isophthalonitrile monohydrate, wherein the compound contains less than 6% by weight of other 4,5-dihydroxy-2-(4-methylbenzyl)isophthalonitrile solvates and crystalline forms thereof.

In one embodiment, the present disclosure relates to a compound which is crystalline form II of 4,5-dihydroxy-2-(4-methylbenzyl)isophthalonitrile monohydrate, wherein the compound contains less than 4% by weight of other 4,5-dihydroxy-2-(4-methylbenzyl)isophthalonitrile solvates and crystalline forms thereof.

In one embodiment, the present disclosure relates to a compound which is crystalline form I of 4,5-dihydroxy-2-(4-methylbenzyl)isophthalonitrile anhydrate, wherein the compound has at least peaks at 2θ of about 6.0° and 11.6° in an X-ray powder diffractogram.

In one embodiment, the present disclosure relates to a compound which is crystalline form I of 4,5-dihydroxy-2-(4-methylbenzyl)isophthalonitrile anhydrate, wherein the compound has at least peaks at 2θ of about 6.0° and 15.9° in an X-ray powder diffractogram.

In one embodiment, the present disclosure relates to a compound which is crystalline form I of 4,5-dihydroxy-2-(4-methylbenzyl)isophthalonitrile anhydrate, wherein the compound has at least peaks at 2θ of about 6.0°, 11.6°, 15.9°, 16.5°, 18.10, 21.4°, 26.5° and 27.7° in an X-ray powder diffractogram.

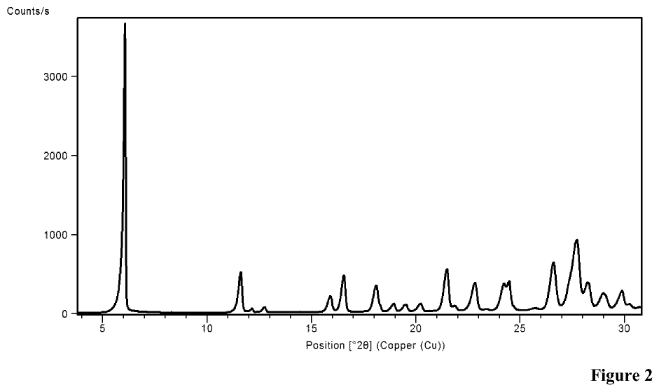

In one embodiment, the present disclosure relates to a compound which is crystalline form I of 4,5-dihydroxy-2-(4-methylbenzyl)isophthalonitrile anhydrate, wherein the compound is characterized by an X-ray powder diffractogram substantially as illustrated in FIG. 2.

In one embodiment, the present disclosure relates to a compound which is crystalline form IV of 4,5-dihydroxy-2-(4-methylbenzyl)isophthalonitrile anhydrate, wherein the compound has at least peaks at 2θ of about 5.6° and 11.7° in an X-ray powder diffractogram.

In one embodiment, the present disclosure relates to a compound which is crystalline form IV of 4,5-dihydroxy-2-(4-methylbenzyl)isophthalonitrile anhydrate, wherein the compound has at least peaks at 2θ of about 5.6° and 15.0° in an X-ray powder diffractogram.

In one embodiment, the present disclosure relates to a compound which is crystalline form IV of 4,5-dihydroxy-2-(4-methylbenzyl)isophthalonitrile anhydrate, wherein the compound has at least peaks at 2θ of about 5.6°, 11.7°, 15.0°, 15.8°, 17.5°, 24.3°, 26.10 and 28.6° in an X-ray powder diffractogram.

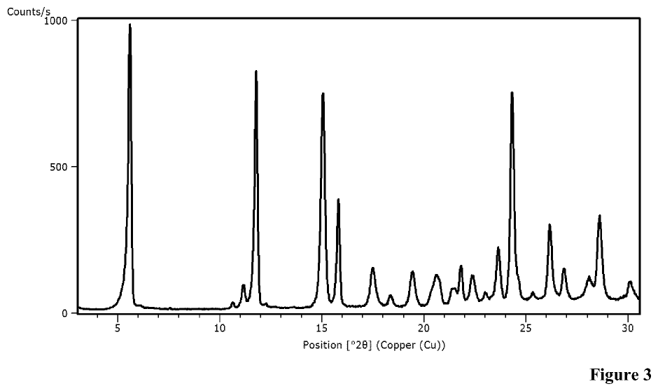

In one embodiment, the present disclosure relates to a compound which is crystalline form IV of 4,5-dihydroxy-2-(4-methylbenzyl)isophthalonitrile anhydrate, wherein the compound is characterized by an X-ray powder diffractogram substantially as illustrated in FIG. 3.

In one embodiment, the present disclosure relates to a compound which is crystalline form III of 4,5-dihydroxy-2-(4-methylbenzyl)isophthalonitrile anhydrate, wherein the compound has at least peaks at 2θ of about 6.1° and 11.8° in an X-ray powder diffractogram.

In one embodiment, the present disclosure relates to a compound which is crystalline form III of 4,5-dihydroxy-2-(4-methylbenzyl)isophthalonitrile anhydrate, wherein the compound has at least peaks at 2θ of about 6.1°, 15.4°, 15.7° and 16.7° in an X-ray powder diffractogram.

In one embodiment, the present disclosure relates to a compound which is crystalline form III of 4,5-dihydroxy-2-(4-methylbenzyl)isophthalonitrile anhydrate, wherein the compound has at least peaks at 2θ of about 6.10, 11.8°, 15.4°, 15.7°, 16.7°, 21.7°, 24.5°, 26.4°, 27.0° and 27.7° in an X-ray powder diffractogram.

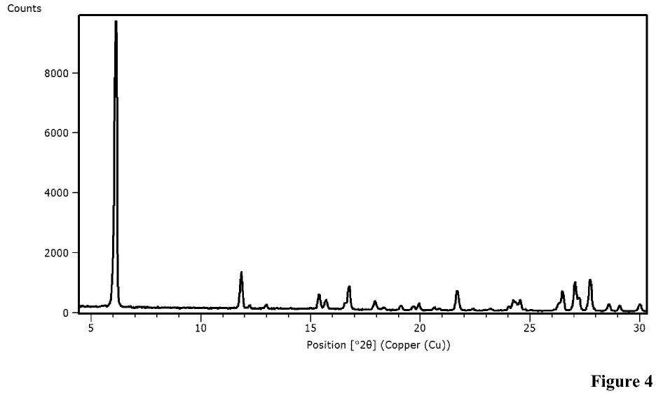

In one embodiment, the present disclosure relates to a compound which is crystalline form III of 4,5-dihydroxy-2-(4-methylbenzyl)isophthalonitrile anhydrate, wherein the compound is characterized by an X-ray powder diffractogram substantially as illustrated in FIG. 4.

In one embodiment, the present disclosure relates to a compound which is crystalline form V of 4,5-dihydroxy-2-(4-methylbenzyl)isophthalonitrile presumably methanol solvate, wherein the compound has at least peaks at 2θ of about 5.9°, 8.1°, 8.7°, 11.8°, 12.0°, 12.8°, 13.8°, 17.4°, 18.3°, 19.2°, 24.2° and 25.4° in an X-ray powder diffractogram.

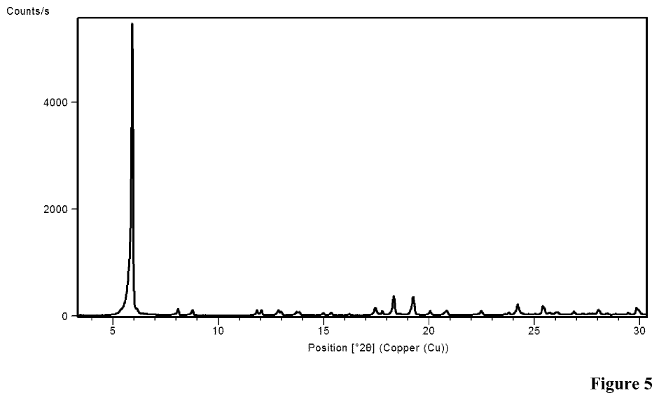

In one embodiment, the present disclosure relates to a compound which is crystalline form V of 4,5-dihydroxy-2-(4-methylbenzyl)isophthalonitrile presumably methanol solvate, wherein the compound is characterized by an X-ray powder diffractogram substantially as illustrated in FIG. 5.

In one embodiment, the present disclosure relates to a compound which is crystalline form VI of 4,5-dihydroxy-2-(4-methylbenzyl)isophthalonitrile monohydrate, wherein the compound has at least peaks at 2θ of about 5.5° and 6.4° in an X-ray powder diffractogram.

In one embodiment, the present disclosure relates to a compound which is crystalline form VI of 4,5-dihydroxy-2-(4-methylbenzyl)isophthalonitrile monohydrate, wherein the compound has at least peaks at 2θ of about 5.5° and 12.9° in an X-ray powder diffractogram.

In one embodiment, the present disclosure relates to a compound which is crystalline form VI of 4,5-dihydroxy-2-(4-methylbenzyl)isophthalonitrile monohydrate, wherein the compound has at least peaks at 2θ of about 5.5°, 6.4°, 8.8°, 12.9°, 19.0°, 23.1°, 25.1° and 27.5° in an X-ray powder diffractogram.

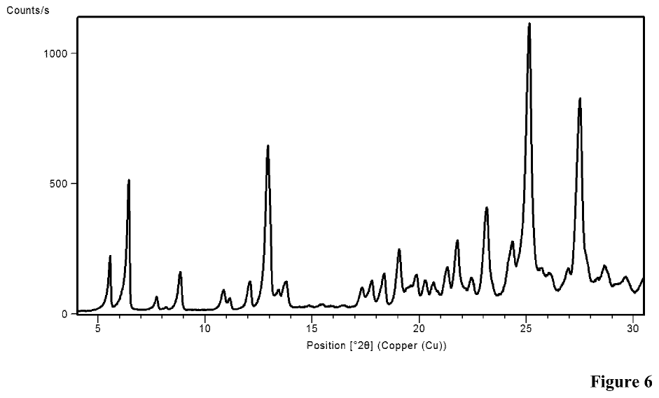

In one embodiment, the present disclosure relates to a compound which is crystalline form VI of 4,5-dihydroxy-2-(4-methylbenzyl)isophthalonitrile monohydrate, wherein the compound is characterized by an X-ray powder diffractogram substantially as illustrated in FIG. 6.

In one embodiment, the present disclosure relates to a compound which is crystalline form VII of 4,5-dihydroxy-2-(4-methylbenzyl)isophthalonitrile presumably methanol solvate, wherein the compound has at least peaks at 2θ of about 6.2°, 8.2°, 8.4°, 11.9°, 18.8°, 23.2° and 25.2° in an X-ray powder diffractogram.

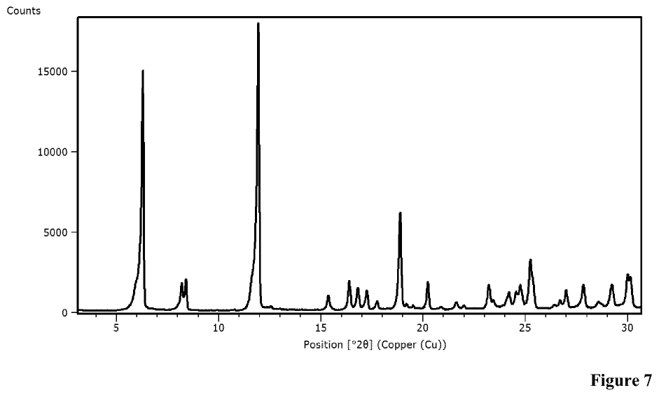

In one embodiment, the present disclosure relates to a compound which is crystalline form VII of 4,5-dihydroxy-2-(4-methylbenzyl)isophthalonitrile presumably methanol solvate, wherein the compound is characterized by an X-ray powder diffractogram substantially as illustrated in FIG. 7.

In one embodiment, the present disclosure relates to a compound which is crystalline form VIII of 4,5-dihydroxy-2-(4-methylbenzyl)isophthalonitrile family of isomorphous solvates T8 presumably ethanol/propan-2-ol and T8a presumably propan-2-ol, wherein the compound has at least peaks at 2θ of about 3.9°, 5.9°, 6.5°, 8.2°, 9.4°, 10.7°, 13.7° and 15.7° in X-ray powder diffractograms.

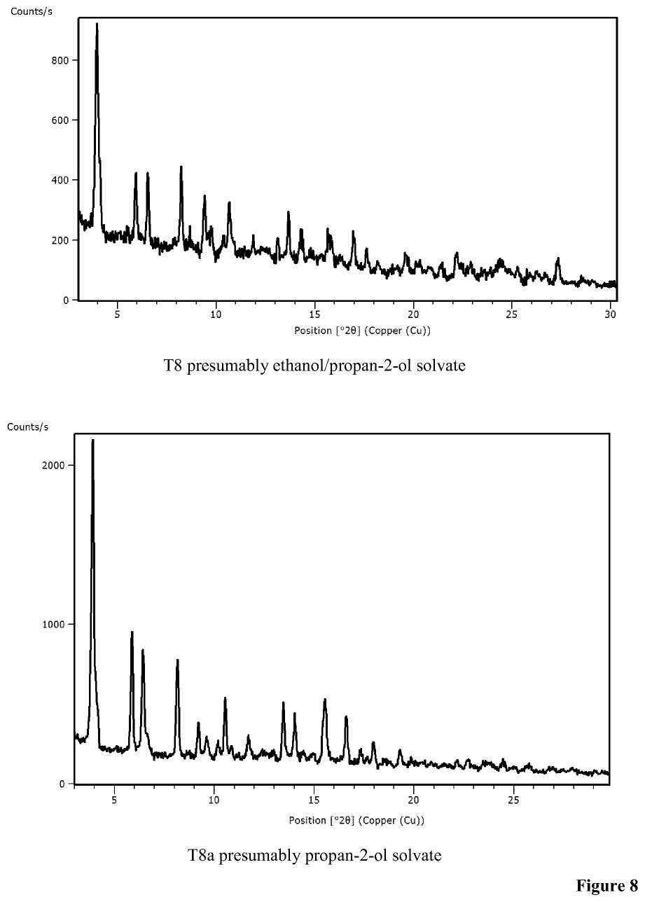

In one embodiment, the present disclosure relates to a compound which is crystalline form VIII of 4,5-dihydroxy-2-(4-methylbenzyl)isophthalonitrile family of isomorphous solvates T8 presumably ethanol/propan-2-ol and T8a presumably propan-2-ol, wherein the compound is characterized by X-ray powder diffractograms substantially as illustrated in FIG. 8.

In one embodiment, the present disclosure relates to a compound which is crystalline form IX of 4,5-dihydroxy-2-(4-methylbenzyl)isophthalonitrile presumably ethanol solvate, wherein the compound has at least peaks at 2θ of about 5.4°, 8.4°, 10.6°, 15.4°, 15.5°, 16.1°, 25.0° and 26.3° in an X-ray powder diffractogram.

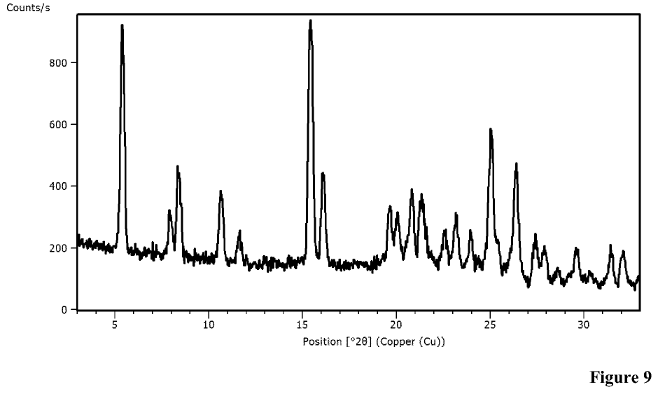

In one embodiment, the present disclosure relates to a compound which is crystalline form IX of 4,5-dihydroxy-2-(4-methylbenzyl)isophthalonitrile presumably ethanol solvate, wherein the compound is characterized by an X-ray powder diffractogram substantially as illustrated in FIG. 9.

In one embodiment, the present disclosure relates to a compound which is crystalline form X of 4,5-dihydroxy-2-(4-methylbenzyl)isophthalonitrile presumably ethanol solvate, wherein the compound has at least peaks at 2θ of about 5.6°, 8.2°, 12.1°, 12.7°, 13.3°, 13.9°, 17.4° and 17.8° in an X-ray powder diffractogram.

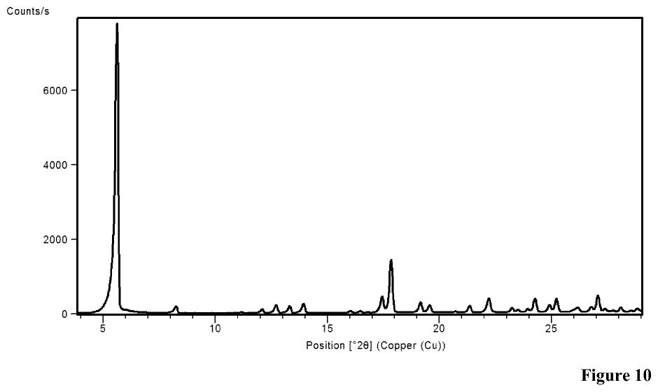

In one embodiment, the present disclosure relates to a compound which is crystalline form X of 4,5-dihydroxy-2-(4-methylbenzyl)isophthalonitrile presumably ethanol solvate, wherein the compound is characterized by an X-ray powder diffractogram substantially as illustrated in FIG. 10.

In one embodiment, the present disclosure relates to a compound which is crystalline form XI of 4,5-dihydroxy-2-(4-methylbenzyl)isophthalonitrile presumably ethanol solvate, wherein the compound has at least peaks at 2θ of about 5.0°, 5.6°, 6.2°, 9.5°, 11.6°, 15.2° and 19.8° in an X-ray powder diffractogram.

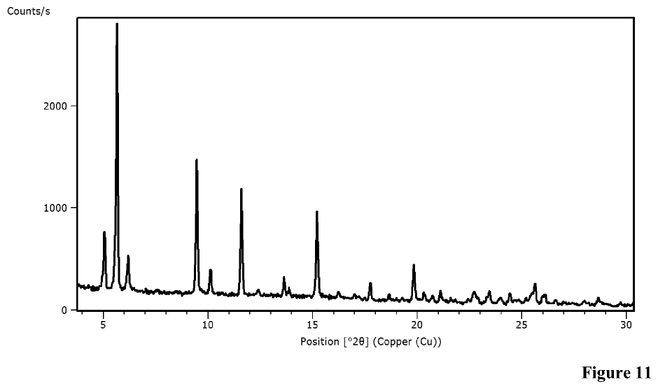

In one embodiment, the present disclosure relates to a compound which is crystalline form XI of 4,5-dihydroxy-2-(4-methylbenzyl)isophthalonitrile presumably ethanol solvate, wherein the compound is characterized by an X-ray powder diffractogram substantially as illustrated in FIG. 11.

A person skilled in the art recognizes that the position of peaks in X-ray powder diffractograms can be subject to variation of ±0.2° 2θ depending on various factors, such as temperature, concentration and instrumentation used. Therefore, the position of peaks is referred to herein as being at “about” specific values.

In one embodiment, the present disclosure relates to a process for the preparation of crystalline form II of 4,5-dihydroxy-2-(4-methylbenzyl)isophthalonitrile monohydrate comprising the steps of

a) mixing acetonitrile, aluminium chloride, sodium iodide and 4-hydroxy-5-methoxy-2-(4-methylbenzyl)isophthalonitrile;

b) heating the mixture obtained in step a) to 35-55° C.;

c) cooling the mixture to 5-25° C.;

d) adding water, HCl and sodium sulphite to the mixture;

e) separating off the aqueous phase from the mixture obtained in step d);

f) adding water, sodium chloride, sodium sulphite and HCl to the organic phase obtained in step e);

g) separating off the aqueous phase from the mixture obtained in step f);

h) concentrating the organic phase obtained in step g);

i) adding ethanol to the residue obtained in step h);

j) optionally concentrating the mixture obtained in step i);

k) adding water to the mixture obtained in step i) or adding water and optionally ethanol to the residue obtained in step j);

l) cooling the mixture obtained in step k) to −10-10° C.;

m) isolating the crystalline product obtained in step l); and

n) drying the crystalline product.

In one embodiment, the mixture in step b) is heated to about 45° C.

In one embodiment, the mixture in step c) is cooled to about 15° C.

In one embodiment, the mixture in step l) is cooled to about 0° C.

In one embodiment, the cooling in step l) is carried out in 5-13 h.

In one embodiment, the cooling in step l) is carried out in 7-11 h.

In one embodiment, the cooling in step l) is carried out in about 9 h.

In one embodiment, the drying in step n) is carried out under reduced pressure at 30-45° C.

In one embodiment, the drying in step n) is carried out under reduced pressure at 35-40° C.

The terms employed herein have the meanings indicated below.

The term “solvate”, as employed herein, refers to solid non-covalent combinations of a solvent and a solute. The solvent can be an organic solvent or water. When the solvent is water, the solvate can also be referred to as hydrate. Thus, the term “hydrate”, as employed herein, refers to solid non-covalent combinations of water and a solute.

The term “form II monohydrate”, as employed herein, refers to crystalline form II of 4,5-dihydroxy-2-(4-methylbenzyl)isophthalonitrile monohydrate.

The term “form I anhydrate”, as employed herein, refers to crystalline form I of 4,5-dihydroxy-2-(4-methylbenzyl)isophthalonitrile anhydrate.

The term “form IV anhydrate”, as employed herein, refers to crystalline form IV of 4,5-dihydroxy-2-(4-methylbenzyl)isophthalonitrile anhydrate.

The term “form III anhydrate”, as employed herein, refers to crystalline form III of 4,5-dihydroxy-2-(4-methylbenzyl)isophthalonitrile anhydrate.

Form I anhydrate, form III anhydrate and form IV anhydrate are converted to form II monohydrate at a relative humidity (RH) above 30%. Form II monohydrate is stable within a reasonable humidity range and enables adequate polymorphic and solvatomorphic control in the manufacture of pharmaceutical products.

Wet granulation is commonly used in the manufacture of pharmaceutical products. Form II monohydrate is suitable for use in wet granulation.

Purity can be assessed with a method known in the art. Suitable methods include, but are not limited to, gas chromatography, column chromatography, liquid chromatography, high-pressure liquid chromatography, thin layer chromatography, mass spectrometry and high-resolution mass spectrometry.

The crystalline forms of the 4,5-dihydroxy-2-(4-methylbenzyl)isophthalonitrile solvates of the present disclosure are useful as medicaments and can be formulated into pharmaceutical dosage forms, such as tablets, capsules, powders or suspensions, by mixing with pharmaceutical excipients known in the art. Suitable excipients include, but are not limited to, fillers, binders, disintegrating agents, lubricants, solvents, gel forming agents, emulsifiers, stabilizers, colourants and preservatives.

The present disclosure will be explained in more detail by the following examples. The examples are meant for illustrating purposes only and do not limit the scope of the invention defined in the claims.

The abbreviations have the meanings indicated below.
HPLC high-performance liquid chromatography
XRPD X-ray powder diffraction

XRPD measurements were performed according to Method 1, Method 2 or Method 3 described below.

Method 1

XRPD patterns were obtained on a PANalytical X'Celerator θ-θ diffractometer with CuKα radiation (40 kV, 30 mA). The diffractometer was operated in reflection mode. The measurements were performed in the range of 3°-40° 2θ. 100-300 mg of the powder was placed in the sample holder and the surface was pressed.

Method 2

XRPD patterns were obtained on a D8 Advance Bruker AXS θ-2θ diffractometer with CuKα radiation (40 kV, 30 mA). The diffractometer was operated in reflection mode. The measurements were performed in the range of 3°-33° 2θ in steps of 0.02°. Small sample amounts (approximately 5 mg) were first placed in the centre of a metal, low background sample holder and then gently spread to a thin layer with a glass rod.

Method 3

XRPD patterns were obtained on a PANalytical X'Celerator θ-θ diffractometer with CuKα radiation (40 kV, 30 mA). The diffractometer was operated in reflection mode. The measurements were performed in the range of 3°-40° 2θ. Small sample amounts (approximately 3-5 mg) were first placed in the centre of a zero-background sample holder and then gently spread to a thin layer.

### Example 1: Crystalline Form II of 4,5-dihydroxy-2-(4-methylbenzyl)isophthalonitrile monohydrate

Acetonitrile (56 ml), aluminium chloride (8 g) and sodium iodide (9.5 g) were charged. 4-hydroxy-5-methoxy-2-(4-methylbenzyl)isophthalonitrile (10 g) was added. The mixture was heated to 45° C., stirred for 4 h and cooled to 15° C. Water (60 ml) and 30% HCl (15 ml) were added slowly at 15° C. Sodium sulphite (2 g) was added and the mixture was stirred for 90 min at 22° C. The phases were allowed to settle and the aqueous phase was separated off. Water (30 ml), sodium chloride (3 g), sodium sulphite (1 g) and 30% HCl (1.5 ml) were added. The mixture was stirred for 1 h at 22° C. and the phases were allowed to settle. The aqueous phase was separated off. Solvents were distilled off under atmospheric pressure until the volume of the residue was 20 ml. Ethanol (80 ml) was added and the distillation was continued until the volume of the residue was 30 ml. The residue was cooled to 70° C. and ethanol (16 ml) and water (65 ml) were added. The mixture was cooled to 0° C. in 9 h and stirred for at least 1 h at 0° C. The crystalline product was filtered and washed with water (15 ml). The product was dried under reduced pressure at 35-40° C. in an agitated dryer. The yield was 92.5% and the HPLC purity 99.5%. The X-ray powder diffractogram of crystalline form II of 4,5-dihydroxy-2-(4-methylbenzyl)isophthalonitrile monohydrate recorded according to Method 1 is shown in FIG. 1.

### Example 2: Crystalline Form I of 4,5-dihydroxy-2-(4-methylbenzyl)isophthalonitrile anhydrate

Form II monohydrate powder (1 g) was dried at 40-60° C. by dry nitrogen gas flow for 3 weeks. The yield was about 95%. The X-ray powder diffractogram of crystalline form I of 4,5-dihydroxy-2-(4-methylbenzyl)isophthalonitrile anhydrate recorded according to Method 1 is shown in FIG. 2.

### Example 3: Crystalline Form IV of 4,5-dihydroxy-2-(4-methylbenzyl)isophthalonitrile anhydrate

Form II monohydrate powder (1000 mg) was placed in a vacuum oven for 5 h at 120° C. The material was cooled to room temperature in an exsiccator with silica gel. The yield was about 95%. The X-ray powder diffractogram of crystalline form IV of 4,5-dihydroxy-2-(4-methylbenzyl)isophthalonitrile anhydrate recorded according to Method 1 is shown in FIG. 3.

### Example 4: Crystalline Form III of 4,5-dihydroxy-2-(4-methylbenzyl)isophthalonitrile anhydrate

A solution (81.6 mg/ml) of form IV anhydrate in ethyl acetate was prepared at room temperature. The solution was added to heptane pre-cooled to 5° C. The solution/heptane volume ratio was 1:4. The product was obtained after slow evaporation in an opened vial. The X-ray powder diffractogram of crystalline form III of 4,5-dihydroxy-2-(4-methylbenzyl)isophthalonitrile anhydrate recorded according to Method 2 is shown in FIG. 4.

### Example 5: Crystalline Form V of 4,5-dihydroxy-2-(4-methylbenzyl)isophthalonitrile Presumably methanol solvate

Form IV anhydrate powder (100 mg) was placed in a 2-ml glass vessel. 1 ml of methanol/water (volume ratio 75:15) was added using pipette. The slurry was left in the hermetically closed vessel overnight. The solid was harvested and left for drying under hood for 30 min. The product obtained is presumably methanol solvate. The X-ray powder diffractogram of crystalline form V of 4,5-dihydroxy-2-(4-methylbenzyl)isophthalonitrile presumably methanol solvate recorded according to Method 3 is shown in FIG. 5.

### Example 6: Crystalline Form VI of 4,5-dihydroxy-2-(4-methylbenzyl)isophthalonitrile monohydrate

Form II monohydrate powder (250 mg) was placed in a Retsch ball mill bowl. Several drops of ethanol were added to assist grinding in two steps, with total grinding times of 2 h and ν=30 Hz. After the elapse of experimental grinding time, the bowl was left open for drying. The yield was 80%. The X-ray powder diffractogram of crystalline form VI of 4,5-dihydroxy-2-(4-methylbenzyl)isophthalonitrile monohydrate recorded according to Method 1 is shown in FIG. 6.

### Example 7: Crystalline Form VII of 4,5-dihydroxy-2-(4-methylbenzyl)isophthalonitrile Presumably methanol solvate

The crystalline form was prepared from form IV anhydrate powder (100 mg) with the method of Example 5 using 1 ml of methanol instead of methanol/water (volume ratio 75:15). The product obtained is presumably methanol solvate. The X-ray powder diffractogram of crystalline form VII of 4,5-dihydroxy-2-(4-methylbenzyl)isophthalonitrile presumably methanol solvate recorded according to Method 3 is shown in FIG. 7.

### Example 8: Crystalline Form VIII of 4,5-dihydroxy-2-(4-methylbenzyl)isophthalonitrile Family of Isomorphous Solvates T8 Presumably ethanol/propan-2-ol and T8a Presumably propan-2-ol

Form IV anhydrate powder (19-20 mg) was placed in glass vials. Slurries were prepared by adding 150-250 μl of ethanol/water (83:17 volume-%) or propan-2-ol/water (97.5:2.5 volume-%). The slurries were aged for 2-3 weeks at 40° C. under continuous stirring at atmospheric pressure. If complete dissolution occurred during the ageing time, the suspension state was obtained by slow evaporation in a slightly opened vial. After the ageing time, the solids were harvested. The product obtained is a family of presumably ethanol/propan-2-ol solvate and presumably propan-2-ol solvate. The X-ray powder diffractograms of crystalline form VIII of 4,5-dihydroxy-2-(4-methylbenzyl)isophthalonitrile family of isomorphous solvates T8 presumably ethanol/propan-2-ol and T8a presumably propan-2-ol recorded according to Method 2 is shown in FIG. 8.

### Example 9: Crystalline Form IX of 4,5-dihydroxy-2-(4-methylbenzyl)isophthalonitrile Presumably ethanol solvate

The crystalline form was prepared from form IV anhydrate powder (20.3 mg) with the method of Example 8 using 150 μl of ethanol/water (96.7:3.3 volume-%) instead of ethanol/water (83:17 volume-%) or propan-2-ol/water (97.5:2.5 volume-%). The product obtained is presumably ethanol solvate. The X-ray powder diffractogram of crystalline form IX of 4,5-dihydroxy-2-(4-methylbenzyl)isophthalonitrile presumably ethanol solvate recorded according to Method 2 is shown in FIG. 9.

### Example 10: Crystalline Form X of 4,5-dihydroxy-2-(4-methylbenzyl)isophthalonitrile Presumably ethanol solvate

The crystalline form was prepared from form IV anhydrate powder (100 mg) with the method of Example 5 using 1 ml of ethanol/hexane (volume ratio 50:50) instead of methanol/water (volume ratio 75:15). The product obtained is presumably ethanol solvate. The X-ray powder diffractogram of crystalline form X of 4,5-dihydroxy-2-(4-methylbenzyl)isophthalonitrile presumably ethanol solvate recorded according to Method 2 is shown in FIG. 10.

### Example 11: Crystalline Form XI of 4,5-dihydroxy-2-(4-methylbenzyl)isophthalonitrile Presumably ethanol solvate

Form IV anhydrate powder (15.6 mg) was placed in a 4-ml vial, which was inserted into a 20-ml vessel containing ethanol (900 μl). The closed diffusion system was let to stand at room temperature for 2 weeks. The product obtained is presumably ethanol solvate. The X-ray powder diffractogram of crystalline form XI of 4,5-dihydroxy-2-(4-methylbenzyl)isophthalonitrile presumably ethanol solvate recorded according to Method 2 is shown in FIG. 11.

A person skilled in the art will appreciate that the embodiments described in the present disclosure can be modified without departing from the inventive concept. A person skilled in the art also understands that the present disclosure is not limited to the particular embodiments disclosed but is intended to also cover modifications of the embodiments that are within the spirit and scope of the present disclosure.

1. A compound which is crystalline form II of 4,5-dihydroxy-2-(4-methylbenzyl)isophthalonitrile monohydrate, wherein the compound has at least peaks at 2θ of about 6.1° and 11.2° in an X-ray powder diffractogram.

2. A compound which is crystalline form II of 4,5-dihydroxy-2-(4-methylbenzyl)isophthalonitrile monohydrate, wherein the compound has at least peaks at 2θ of about 6.1° and 14.7° in an X-ray powder diffractogram.

3. The compound according to claim 1, wherein the compound has at least peaks at 2θ of about 6.1°, 11.2°, 14.7°, 16.2°, 21.8°, 24.0°, 26.2° and 26.9° in an X-ray powder diffractogram.

4. The compound according to claim 1, wherein the compound is characterized by an X-ray powder diffractogram substantially as illustrated in FIG. 1.

5. The compound according to claim 1, wherein the compound contains less than 25% by weight of other 4,5-dihydroxy-2-(4-methylbenzyl)isophthalonitrile solvates and crystalline forms thereof.

6. The compound according to claim 5, wherein the compound contains less than 10% by weight of other 4,5-dihydroxy-2-(4-methylbenzyl)isophthalonitrile solvates and crystalline forms thereof.

7. The compound according to claim 6, wherein the compound contains less than 6% by weight of other 4,5-dihydroxy-2-(4-methylbenzyl)isophthalonitrile solvates and crystalline forms thereof.

8. The compound according to claim 7, wherein the compound contains less than 4% by weight of other 4,5-dihydroxy-2-(4-methylbenzyl)isophthalonitrile solvates and crystalline forms thereof.

9. A process for the preparation of a compound according to claim 1 comprising the steps of;
a) mixing acetonitrile, aluminium chloride, sodium iodide and 4-hydroxy-5-methoxy-2-(4-methylbenzyl)isophthalonitrile;
b) heating the mixture obtained in step a) to 35° C. to 55° C.;
c) cooling the mixture to 5° C. to 25° C.;
d) adding water, HCl and sodium sulphite to the mixture;
e) separating off the aqueous phase from the mixture obtained in step d);
f) adding water, sodium chloride, sodium sulphite and HCl to the organic phase obtained in step e);
g) separating off the aqueous phase from the mixture obtained in step f);
h) concentrating the organic phase obtained in step g);
i) adding ethanol to the residue obtained in step h);
j) optionally concentrating the mixture obtained in step i);
k) adding water to the mixture obtained in step i) or adding water and optionally ethanol to the residue obtained in step j);
l) cooling the mixture obtained in step k) to −10° C. to 10° C.;
m) isolating the crystalline product obtained in step l); and
n) drying the crystalline product.

10. The process according to claim 9, wherein the mixture in step b) is heated to about 45° C.

11. The process according to claim 9, wherein the mixture in step c) is cooled to about 15° C.

12. The process according to claim 9, wherein the mixture in step l) is cooled to about 0° C.

13. The process according to claim 9, wherein the cooling in step l) is carried out in 5-13 h.

14. The process according to claim 13, wherein the cooling in step l) is carried out in 7-11 h.

15. The process according to claim 14, wherein the cooling in step l) is carried out in about 9 h.

16. The process according to claim 9, wherein the drying in step n) is carried out under reduced pressure at 30° C. to 45° C.

17. The process according to claim 16, wherein the drying in step n) is carried out under reduced pressure at 35° C. to 40° C.

18. A pharmaceutical dosage form comprising a compound according to claim 1 and a pharmaceutically acceptable excipient.

19. A pharmaceutical dosage form comprising a compound according to claim 2 and a pharmaceutically acceptable excipient.

20. The compound according to claim 2, wherein the compound has at least peaks at 2θ of about 6.1°, 11.2°, 14.7°, 16.2°, 21.8°, 24.0°, 26.2° and 26.9° in an X-ray powder diffractogram.

21. The compound according to claim 2, wherein the compound is characterized by an X-ray powder diffractogram substantially as illustrated in FIG. 1.

22. The compound according to claim 2, wherein the compound contains less than 25% by weight of other 4,5-dihydroxy-2-(4-methylbenzyl)isophthalonitrile solvates and crystalline forms thereof.

23. The compound according to claim 22, wherein the compound contains less than 10% by weight of other 4,5-dihydroxy-2-(4-methylbenzyl)isophthalonitrile solvates and crystalline forms thereof.

24. The compound according to claim 23, wherein the compound contains less than 6% by weight of other 4,5-dihydroxy-2-(4-methylbenzyl)isophthalonitrile solvates and crystalline forms thereof.

25. The compound according to claim 24, wherein the compound contains less than 4% by weight of other 4,5-dihydroxy-2-(4-methylbenzyl)isophthalonitrile solvates and crystalline forms thereof.
